(19) United States
(12) Patent Application Publication
(10) Pub. No.: US 2014/0081110 A1
(43) Pub. Date: Mar. 20, 2014

(54) ELECTROPHYSIOLOGICAL DIAGNOSIS AND TREATMENT FOR ASTHMA

(71) Applicant: Edwin C. Jesudason, Liverpool (GB)

(72) Inventor: Edwin C. Jesudason, Liverpool (GB)

(73) Assignees: University of Liverpool, Liverpool (GB); Children's Hospital Los Angeles, Los Angeles, CA (US)

(21) Appl. No.: 14/123,732

(22) PCT Filed: Jun. 4, 2012
(86) PCT No.: PCT/US12/40770

§ 371 Date: Dec. 9, 2013

Related U.S. Application Data
(60) Provisional application No. 61/493,306, filed on Jun. 3, 2011.

(51) Int. Cl. A61B 5/04; A61B 1/267; A61B 1/018; A61N 1/36; A61B 18/14; A61B 5/00; A61B 5/20; A61B 1/307; A61B 5/08; A61B 18/18

(52) U.S. Cl. CPC A61B 5/04; A61B 5/08; A61B 1/2676; A61B 1/018; A61B 18/18; A61B 18/1492; A61B 5/7282; A61B 5/201; A61B 1/307; A61B 5/742; A61N 1/3601
USPC 600/373

(57) ABSTRACT

The invention provides methods for diagnosing and treating asthma in subjects in need thereof but can be applied to other conditions where abnormal electrophysiology underlies dysfunctional organ contractility.

### FIELD OF INVENTION

The invention is directed to methods and systems for using electrophysiology to diagnose and treat abnormal smooth muscle contractility resulting in, amongst other diseases, asthma.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Asthma is amongst the most common chronic diseases of US children, and is responsible for innumerable primary care consultations, hospital admissions, intensive care stays and premature deaths. Steroids and bronchodilators remain mainstays of current palliative therapy based on a paradigm that unites airway inflammation, allergy, bronchospasm and remodeling. In vitro research has not fully captured airway contractility in vivo. Recognizing airway contractility in prenatal airway, the inventor proposes a new paradigm of electrophysiological (EP) diagnosis and treatment of asthma. In the instant paradigm, asthma is an intrinsic dysrhythmia of airway contractility secondarily influenced by allergy and inflammation. Rhythmicity of airway contraction is a prenatal norm and may persist postnatally in an abnormal form in asthma. Rhythmicity in asthma is already visible in diurnal symptom variations. Moreover normal fluctuations of airway calibers (homeokinesis) are exaggerated in asthma. The suggestion herein that asthma is an intrinsic airway problem is supported by persistence of asthma in lungs transplanted into non-asthmatics and non-recurrence in asthmatics transplanted with non-asthmatic lungs. Similarly the failure of lung denervation to cure asthma supports an intrinsic problem.

Having observed a pacemaker hierarchy prenatally (where the pacemaker drives the rhythm of observed airway contractions and may comprise the pulmonary epithelium, vasculature, the airway smooth muscle and/or nerves), the proposal is that intrinsic large airway pacemakers regulate small and medium sized airway contractility in healthy and asthmatic subjects. Thus the inventor's novel approach is electrophysiological mapping to diagnose asthma by locating aberrant pacemaker activity and treat asthma and possibly cure it, by radiofrequency ablation of aberrant pacemaker sites and/or interruption of propagating smooth muscle contractility between proximal and distal airways. For refractory disease, long-term electrophysiological monitoring of the airways by implanted device could warn of, and pre-empt, impending attacks.

Bronchial thermoplasty is a promising asthma therapy based on indiscriminate airway smooth muscle (ASM) ablation in numerous large and medium-sized airways (despite concerns about long-term peri-bronchial fibrosis and consequently untreatable airway obstruction). How it might relieve symptoms also remains unclear since it is the smaller airway constriction that characterizes asthma. The inventor's proposal provides the missing mechanism: thermoplasty is unwittingly ablating proximal airway pacemakers that regulate distal airway contractility (rendering them quiescent).

### SUMMARY OF THE INVENTION

Contrary to current drug therapy for asthma or indeed bronchial thermoplasty to destroy airway smooth muscle in severe drug refractory disease, the instant invention uses airway electrophysiology to diagnose asthma and to target thermal ablation to pacemaker sites, thereby limiting the therapeutic damage inflicted and hence allowing the therapy to be used for all disease stages with the prospect of long term treatment and possible cure. In addition, the use of airway electrophysiology to diagnose and treat asthma allows monitoring of the airway electrophysiology using implantable devices in patients with chronic asthma. This approach permits attacks to be detected early and pre-empted using application of appropriate currents to render the airway smooth muscle quiescent.

### BRIEF DESCRIPTION OF FIGURES

FIG. 1 depicts the concept of the instant invention.

FIG. 2 depicts (A) Airway peristalsis (AP) induces fluid flux:Far left: Fluid flux in prenatal airway lumen (dark) seen as debris moves (arrows) in serial stills (top to bottom). With airway contraction (boxed in middle stack) during AP wave (from our ref 6). (B) Ca2+ waves arise from pacemaker foci seen against time (s). Stacked images of Fluo-4 loaded airway show Ca2+ wave propagation from initiation sites (asterisk) in trachea (top row) and R bronchus (bottom row).

FIG. 3 depicts spatial mapping. The bronchoscope allows the EP catheter to be placed under vision as desired to obtain a map of signal by location. This technique allows exploration of the sites of maximal prenatal pacemaker activity and the airway generations accessed during bronchial thermoplasty. In subsequent studies described below, the spatial mapping technique allows focal electrophysiological stimulation to be applied to the airway in order to determine its effects on strength and spread of airway contractility.

FIG. 4 depicts temporal mapping. The electrodes are placed using thoracoscopic guidance on the outer surface of the airway (distal tracheal positioning is illustrated for example here). These electrodes are connected to the pacemaker device with sensing and pacing modes. The device is secured in a subcutaneous pouch (as cardiac pacemakers are). This technique allows exploration of whether prenatal pacemaker periodicities are apparent over longer time scales postnatally and to measure airway EP during daily living and without anesthesia or instrumentation of the airway.

FIG. 5 depicts an electropulmonogram. The lower trace shows an electropulmonogram from the right sub carina, a pacemaker region (defined in the inventor's published prenatal work). Note the downward deflections that do not correspond to the simultaneous EKG (above) and which are separated by a time interval shown by the dashed bar, that is not straightforwardly related to the EKG since it spans more than 2 heart beats. The activity in the lower trace is also much faster than breathing movements (see time scale bottom right).

FIG. 6 depicts an electropulmonogram. The lower trace shows an electropulmonogram from the left sub carina, a region defined during our published prenatal work as having lesser pacemaker activity. Note we did not note the same activity seen in the R sub carina. During the temporal mapping we will have a longer recording time to see if this area will generate slower rhythms too. The simultaneous EKG is seen in the top trace. Comparison of FIGS. 5 and 6 confirms the spatial variation in the electropulmonogram that supports focal pacemaker zones within the airway.

FIG. 7 depicts an electropulmonogram. The lowest trace shows pacing signal that we applied to the proximal trachea. The period of this rhythmic input is given by the dashed line and its rhythmic output can be seen in the middle trace in the resulting electropulmonogram from the trachea lower down its anatomical course. It demonstrates that the paced excitation is transmitted from proximal to distal as suggested in our prenatal work. The middle trace also shows separate effects of the EKG rhythm (simultaneous heart recording seen above). The period of this rhythm is illustrated by the double arrowed lines in the EKG and the tracheal electropulmonogram seen in the middle. This shows that these separate signals can be readily distinguished within the electropulmonogram on the basis of their underlying rhythm.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Electropulmonogram” as used herein is a recording of electrical activity from the lung over time. This can be achieved using an electrophysiology catheter applied directly to the airway to measure the depolarization and repolarization associated with normal and abnormal airway contractility during homeokinesis. This may be analogous to the electocardiographic recordings achieved during cardiac catheterization. Alternately, electropulmonogram may be recordable using skin surface electrodes in conjunction with the appropriate software to screen out the other background signals (like electrogastrography).

“Electroureterogram” as used herein is a recording of electrical activity from the ureter over time. This can be achieved using an electrophysiology catheter applied directly to the ureter via an ureteroscope to measure the depolarization and repolarization associated with normal and abnormal ureteric contractility. This may be analogous to the electocardiographic recordings achieved during cardiac catheterization. Alternately, electroureterogram may be recordable using skin surface electrodes in conjunction with the appropriate software to screen out the other background signals (like electrogastrography).

“Pacemaker” as used herein refers to the capacity of a cell or groups of cells to produce the rhythmic behaviors seen in prenatal airway. Such cells may vary in origin and may comprise, for example, airway smooth muscle cells, interstitial cells, nerve cells and/or epithelial cells in conjunction with other cell types. The pacemaker rhythm may be derived by a single command cell or by the emergent effects of a group of diverse cells. The locations of these pacemakers may be described as proximal (i.e. in the trachea and main bronchi), middle (i.e in the lobar bronchi and sub-bronchi beyond) and distal (i.e in the small airways at and beyond the limit of current routine fibreoptic bronchoscopy). This distinction is of relevance to the anatomical locations from which the inventor has obtained recordings (see below).

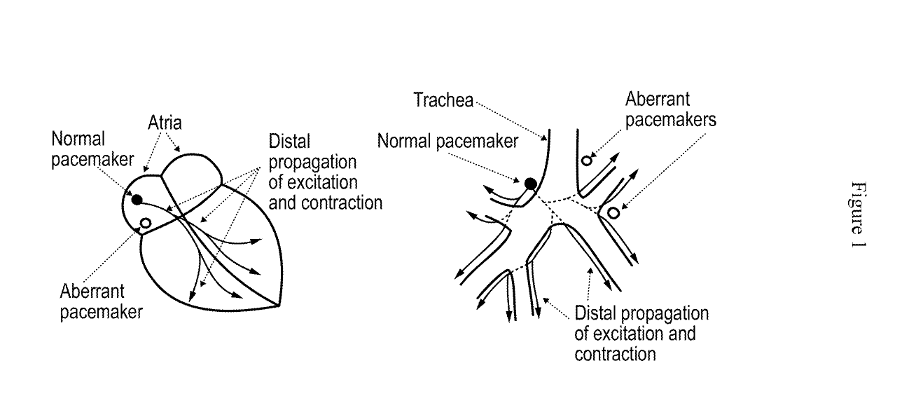

The inventor hypothesizes that asthma may be an airway smooth muscle (ASM) dysrhythmia, that like cardiac dysrhythmias has triggers (e.g. allergens in asthma), but which is treatable and/or curable by ASM pacemaker ablation. The heart (FIG. 1, left) features a pacemaker hierarchy that is established in utero and persists through life. A proximal pacemaker dominates and regulates downstream excitation and contraction. The inventor demonstrated a similar pacemaker hierarchy in proximal prenatal airway that drives downstream airway contractility before birth (FIG. 1, right), and postulates that these pacemaker areas persist and are responsible for normal fluctuation of airway contractility observed after birth (homeokinesis). Like the heart therefore, the lung can be susceptible to abnormal airway smooth muscle (ASM) contractility due to aberrant pacemaker activity. In FIG. 1, normal pacemakers are active and the aberrant ones are quiescent. Electrophysiological treatment and/or cure for asthma would involve targeted ablation of abnormal pacemaker sites. The normal airway electrophysiology (EP) and thermoplasty's impact in vivo and use airway electrophysiology to detect and modulate airway contractility in vivo are ascertained herein.

The instant invention represents the first use of: (1) airway electrophysiology to generate an ‘electropulmonogram’, (2) the electrophysiology data to distinguish the asthmatic airway from a normal airway, (3) the electrophysiology data to define airway pacemaker areas, (4) the electrophysiology information to target pacemaker ablation for the treatment or the potentially curative treatment of asthma at all stages, and (5) implantable devices to detect and treat episodes of abnormal electrophysiology in chronic asthma.

### Diagnostic Methods of the Invention

### Asthma Diagnosis and Treatment

The invention is directed to methods for diagnosing asthma in a subject in need thereof. The method comprises inserting a bronchoscope in the lung of the subject, inserting an electrophysiology catheter through the bronchoscope, obtaining an electropulmonogram comprising electrical impulses from the airway and surrounding lung and diagnosing the presence or absence of asthma in the subject wherein a difference in the electropulmonogram (whether spontaneous or in response to EP stimulation) between the subject and control subjects is indicative of asthma in the subject. In various embodiments of the invention, the electropulmonogram of normal and asthmatic subjects may be distinguishable by their frequency of spontaneous depolarization/hyperpolarisation and/or the morphology of the waveform (for example, the speed of depolarization, time to half-maximal depolarization/hyperpolarisation, the extent of any plateau phase, the speed of return of polarization to baseline, and/or the speed and size of any pacemaker current; further measures of wave morphology are given in Table 1). The frequencies of spontaneous depolarization/hyperpolarisation and the various morphologies of the waveform will be apparent to a person of skill in the art. These differences in the electropulmonogram between the subject and control subjects may be accentuated by administering challenges that promote bronchospasm and reduced by bronchorelaxant medication. Examples of challenges that promote bronchospasm include cholinergic agonists including but not limited to methacholine. In some embodiments, methacholine is administered according to the American Thoracic Society guidelines on methacholine challenge testing (Am J Respir Crit Care Med Vol 161. pp 309-329, 2000). For example, an advised dose schedule is given in Table 4 of the aforementioned guidelines. The doses used can vary from low doses in those with asthma such as 0.031 mg of methacholine per ml of normal saline, up to 16 mg/ml in normal subjects. The UK guidance on asthma indicates that in a normal airway a methacholine dose of >8 mg/ml is required to reduce the Forced Expiratory Volume in 1 Second (FEV1) by 20% (British Guideline on the Management of Asthma, Revised January 2012). Examples of bronchorelaxant medications include cholinergic antagonists (e.g. ipratroprium bromide, typically 500 micrograms (range 100 micrograms-1000 micrograms) in 2.5 ml saline via nebulizer), theophylline (5 mg/kg loading does with 5-16 mg/kg/day maintenance dose, not exceeding 900 mg/day) or beta 2 adrenoceptor agonists (e.g. salbutamol, typically 5 mg (range 1-10 mg)/2.5 ml saline or other diluent).

The invention also provides methods for diagnosing abnormal airway contractility in a subject in need thereof. The method comprises inserting a bronchoscope in the lung of the subject, inserting an electrophysiology catheter through the bronchoscope, obtaining an electropulmonogram comprising electrical impulses from the lung and diagnosing abnormal airway contractility wherein the difference in the electropulmonogram (whether spontaneous or in response to EP stimulation) between the subject and control subjects is indicative of abnormal airway contractility in the subject. In an embodiment, abnormal airway contractility is indicative of asthma in the subject. In various embodiments of the invention, the electropulmonogram of normal and asthmatic subjects may be distinguishable by their frequency of spontaneous depolarization/hyperpolarisation and the morphology of the waveform (for example, the speed of depolarization, time to half-maximal depolarization/hyperpolarisation, the extent of any plateau phase, the speed of return of polarization to baseline, and/or the speed and size of any pacemaker current; further measures of wave morphology are given in Table 1). The frequencies of spontaneous depolarization/hyperpolarisation and the various morphologies of the waveform will be apparent to a person of skill in the art. These differences in the electropulmonogram (whether spontaneous or in response to EP stimulation) between the subject and control subjects may be accentuated by administering challenges that promote bronchospasm and reduced by bronchorelaxant medication. Examples of challenges that promote bronchospasm include cholinergic agonists including methacholine. In some embodiments, methacholine is administered according to the American Thoracic Society guidelines on methacholine challenge testing (Am J Respir Crit Care Med Vol 161. pp 309-329, 2000). For example, an advised dose schedule is given in Table 4 of the American Thoracic Society guidelines. The doses used can vary from low doses in those with asthma such as 0.031 mg of methacholine per ml of normal saline, up to 16 mg/ml in normal subjects. The UK guidance on asthma indicates that in a normal airway a methacholine dose of >8 mg/ml is required to reduce the Forced Expiratory Volume in 1 Second (FEV1) by 20% (British Guideline on the Management of Asthma, Revised January 2012). Examples of bronchorelaxant medications include cholinergic antagonists (e.g. ipratroprium bromide, typically 500 micrograms (range 100 micrograms-1000 micrograms) in 2.5 ml saline via nebulizer), theophylline (5 mg/kg loading does with 5-16 mg/kg/day maintenance dose, not exceeding 900 mg/day) or beta 2 adrenoceptor agonists (e.g. salbutamol, typically 5 mg (range 1-10 mg)/2.5 ml saline or other diluent).

The invention further provides a method for detecting abnormal pacemakers in the lung in a subject in need thereof. The method comprises inserting a standard flexible bronchoscope in the airways of the subject, inserting a standard multipolar electrophysiology catheter through the bronchoscope, obtaining an electropulmonogram comprising electrical impulses from the lung and detecting abnormal pacemakers in the subject wherein a difference in the electropulmonogram (whether spontaneous or in response to EP stimulation) between the subject and control subjects is indicative of abnormal pacemakers in the subject. In an embodiment, abnormal pacemakers are indicative of asthma in the subject. In various embodiments of the invention, the electropulmonogram of normal and asthmatic subjects may be distinguishable by their frequency of spontaneous depolarization/hyperpolarisation and the morphology of the waveform (for example, the speed of depolarization, time to half-maximal depolarization/hyperpolarisation, the extent of any plateau phase, the speed of return of polarization to baseline, and/or the speed and size of any pacemaker current; further measures of wave morphology are given in Table 1). The frequencies of spontaneous depolarization/hyperpolarisation and the various morphologies of the waveform will be apparent to a person of skill in the art (see Table 1). These differences in the electropulmonogram between the subject and control subjects may be accentuated by administering challenges that promote bronchospasm and reduced by bronchorelaxant medication. Examples of challenges that promote bronchospasm include cholinergic agonists including methacholine. In some embodiments, methacholine is administered according to the American Thoracic Society guidelines on methacholine challenge testing (Am J Respir Crit Care Med Vol 161. pp 309-329, 2000). For example, an advised dose schedule is given in Table 4 of the American Thoracic Society guidelines. The doses used can vary from low doses in those with asthma such as 0.031 mg of methacholine per ml of normal saline, up to 16 mg/ml in normal subjects. The UK guidance on asthma indicates that in a normal airway a methacholine dose of >8 mg/ml is required to reduce the Forced Expiratory Volume in 1 Second (FEV1) by 20% (British Guideline on the Management of Asthma, Revised January 2012). Examples of bronchorelaxant medications include cholinergic antagonists (e.g. ipratroprium bromide, typically 500 micrograms (range 100 micrograms-1000 micrograms) in 2.5 ml saline via nebulizer), theophylline (5 mg/kg loading does with 5-16 mg/kg/day maintenance dose, not exceeding 900 mg/day) or beta 2 adrenoceptor agonists (e.g. salbutamol, typically 5 mg (range 1-10 mg)/2.5 ml saline or other diluent).

Also provided herein is a method for diagnosing asthma in a subject in need thereof comprising obtaining the electrical impulses of the lung using a means for sensing the electrical impulses, processing and multiplexing the electrical impulses received from the means for sensing to create a two dimensional waveform and comparing the two dimensional waveform to other waveforms contained within a database in a microprocessor which is comprised of waveforms of patients that have asthma. In one embodiment, the two dimensional waveform is an electropulmonogram. The method further comprises the steps of displaying on a screen a computer generated image of the patient's lung in which any areas of airway constriction are identified and displaying on a screen the two dimensional waveform representing the electrical impulses of the patient's lung.

The invention further provides a challenge test for diagnosing asthma in a subject in need thereof comprising administering a bronchoconstrictor drug to the subject (for example cholinergic agonists including methacholine; In some embodiments, methacholine is administered according to the American Thoracic Society guidelines on methacholine challenge testing (Am J Respir Crit Care Med Vol 161. pp 309-329, 2000). An advised dose schedule is given in Table 4 of the aforementioned American Thoracic Society guidelines. The doses used can vary from low doses in those with asthma such as 0.031 mg of methacholine per ml of Normal saline, up to 16 mg/ml in normal subjects. The UK guidance on asthma indicates that in normal airway a methacholine dose of >8 mg/ml is required to reduce the Forced Expiratory Volume in 1 Second (FEV1) by 20% (British Guideline on the Management of Asthma, Revised January 2012) and then obtaining an electropulmonogram (EPG). The EPG from the subject is compared to the EPG of a control subject who has also been challenged with a bronchoconstrictor drug. A difference in EPG between the subject and the control subject and their EPG responses to bronchoconstrictor and bronchodilator is indicative of asthma. In various embodiments, the electropulmonograms of subject and the control subject may be distinguishable by their frequency of spontaneous depolarization/hyperpolarisation and the morphology of the waveform (for example, the speed of depolarization, time to half-maximal depolarization/hyperpolarisation, the extent of any plateau phase, the speed of return of polarization to baseline, and/or the speed and size of any pacemaker current; further measures of wave morphology are given in Table 1). The frequencies of spontaneous depolarization/hyperpolarisation and the various morphologies of the waveform will be apparent to a person of skill in the art (see Table 1).

In various embodiments, in subjects with asthma, methacholine may be administered as a bronchoconstrictor at any one or more of 0.001 mg to 0.005 mg methacholine per ml of Normal saline, 0.005 mg to 0.010 mg methacholine per ml of Normal saline, 0.010 mg to 0.015 mg methacholine per ml of Normal saline, 0.015 mg to 0.020 mg methacholine per ml of Normal saline, 0.020 mg to 0.025 mg methacholine per ml of Normal saline, 0.025 mg to 0.030 mg methacholine per ml of Normal saline, 0.030 mg to 0.035 mg methacholine per ml of Normal saline, 0.035 mg to 0.04 mg methacholine per ml of Normal saline, 0.04 mg to 0.045 mg methacholine per ml of Normal saline, 0.045 mg to 0.05 mg methacholine per ml of Normal saline, 0.05 mg to 0.06 mg methacholine per ml of Normal saline, 0.06 mg to 0.07 mgmethacholine per ml of Normal saline, 0.07 mg to 0.08 mg methacholine per ml of Normal saline, 0.08 mg to 0.09 mg methacholine per ml of Normal saline, 0.09 mg to 1.0 mg methacholine per ml of Normal saline, 1 mg to 2 mg methacholine per ml of Normal saline, 2 mg to 4 mg methacholine per ml of Normal saline, 4 mg to 6 mg methacholine per ml of Normal saline, 6 mg to 8 mg methacholine per ml of Normal saline, 8 mg to 10 mg methacholine per ml of Normal saline, 10 mg to 12 mg methacholine per ml of Normal saline, 12 mg to 14 mg methacholine per ml of Normal saline, 14 mg to 16 mg methacholine per ml of Normal saline, 16 mg to 20 mg methacholine per ml of Normal saline or a combination thereof. The optimum dosages will be apparent to a person having ordinary skill in the art.

In various embodiments, methacholine may be administered to normal subject as a bronchoconstrictor at any one more of 0.1 to 5 mg/ml of Normal saline, 5 to 6 mg/ml of Normal saline, 6 to 7 mg/ml of Normal saline, 7 to 8 mg/ml of Normal saline, 8 to 10 mg/ml of Normal saline, 10 to 12 mg/ml of Normal saline, 12 to 14 mg/ml of Normal saline, 14 to 16 mg/ml of Normal saline, 16 to 18 mg/ml of Normal saline, 18 to 20 mg/ml of Normal saline, 20 to 25 mg/ml of Normal saline or a combination thereof. The optimum dosages will be apparent to a person having ordinary skill in the art.

In various embodiments, a bronchorelaxant such as ipratroprium bromide may be administered via a nebulizer at any one or more of 100 to 200 micrograms in 2.5 ml saline, 200 to 300 micrograms in 2.5 ml saline, 300 to 400 micrograms in 2.5 ml saline, 400 to 500 micrograms in 2.5 ml saline, 500 to 600 micrograms in 2.5 ml saline, 600 to 700 micrograms in 2.5 ml saline, 700 to 800 micrograms in 2.5 ml saline, 800 to 900 micrograms in 2.5 ml saline, 900 to 1000 micrograms in 2.5 ml saline, or a combination thereof. The optimum dosage will be apparent to a person having ordinary skill in the art.

### Treatment Methods of the Invention

The invention is also directed to methods for treating asthma in a subject in need thereof. In one embodiment, the method comprises detecting abnormal pacemakers using the methods described and using thermal ablation of abnormal pacemakers to control contractility, so as to treat asthma. Specifically, the method comprises detecting abnormal pacemakers by obtaining an electropulmonogram illustrating electrical impulses from one or more lungs of the subject. The electropulmonogram is obtained by inserting a flexible bronchoscope in the one or more lungs of the subject, inserting an electrophysiology catheter through the bronchoscope and measuring the electrical impulses from the one or more lungs using the electrophysiology catheter. The electropulmonogram is analyzed for frequency of spontaneous depolarization and/or hyperpolarization and/or morphology of the waveform. In some embodiments, the differences in the frequency of spontaneous depolarization and/or hyperpolarization and/or morphology of the waveform between the subject and a control subject are indicative of abnormal pacemakers in the lung in the subject. A thermal ablation catheter is inserted into the flexible bronchoscope to target the abnormal pacemaker and thermal ablation is applied to reduce the activity of or destroy the abnormal pacemaker, so as to treat asthma in the subject.

In another embodiment, treating asthma in a subject in need thereof comprises detecting abnormal airway smooth muscle activity (ASM) by the methods described above, inserting a radiofrequency catheter through the bronchoscope to target abnormal airway smooth muscle (ASM) and destroying abnormal smooth muscle using radiofrequency ablation to render distal airway smooth muscle quiescent, so as to treat asthma in the subject. Specifically, the method comprises detecting abnormal rhythmic peristalsis in the subject by obtaining an electropulmonogram illustrating electrical impulses from one or more lungs of the subject. An electropulmonogram is obtained by inserting a flexible bronchoscope in the one or more lungs of the subject, inserting an electrophysiology catheter through the bronchoscope and measuring the electrical impulses from the one or more lungs using the electrophysiology catheter. The electropulmonogram is analyzed for frequency of spontaneous depolarization and/or hyperpolarization and/or morphology of the waveform. In some embodiments differences in the frequency of spontaneous depolarization and/or hyperpolarization and/or morphology of the waveform between the subject and a control subject is indicative of abnormal rhythmic peristalsis in the subject. A radiofrequency catheter is inserted through the flexible bronchoscope to target abnormal airway smooth muscle and the abnormal smooth muscle is focally destroyed and/or inhibited using radiofrequency ablation to render airway smooth muscle quiescent, so as to treat the asthma in the subject.

The invention is also directed to implantable electrodes for subjects with refractory asthma. In one embodiment, the implantable electrodes are programmable airway pacemakers. In another embodiment, the implantable electrodes comprise a sensing electrode such that in the event of an abnormal EPG, a warning is triggered. Therefore in much the same way that cardiac pacemakers can be programmed to detect incipient dysrhythmias, the implanted airway pacemaker devices may monitor for adverse changes in the baseline elctropulmonogram and either transmit an alert such as a text message warning the subject to administer their rescue medication via inhaler, remove themselves from an asthma-prone environment or seek medical help. With understanding of the electropulmonographic disturbance beneath asthma, such implanted devices could not only detect abnormal behavior but via application of e.g. slow and gentle depolarization render the airway quiescent and refractory to contraction.

### Vesicoureteric Reflux Disease

The invention is also directed to methods for diagnosing vesicoureteral reflux in a subject in need thereof. The method comprises inserting a flexible ureteroscope in the ureter of the subject, inserting an electrophysiology catheter through the flexible ureteroscope,

obtaining a spatially mapped electroureterogram comprising electrical impulses from different points along the ureter. This activity is then initially at least correlated with vesicoureteric reflux as demonstrated on voiding cystourethography in the subject wherein a difference in the spatial electroureterogram between the subject and control subject is indicative of vesicoureteral reflux in the subject. Hence the abnormal electroureterograms measured from the vesicoureteric junction and more proximally will enable vesicoureteric reflux to be diagnosed on the basis of abnormal ureteric contractility and the underlying electroureterogram. This then allows implantable pacemakers to detect and counteract such abnormal contraction and signal and to render ureteric peristalsis more efficient at clearing the ureter of urine even if reflux does occur.

### Systems of the Invention

The invention further provides a system for testing a subject for abnormal smooth muscle contractility comprising a microprocessor comprising a signal processor and a pattern recognition processor, a means for conducting the electrical impulses of the subject's lung and communicating the electrical impulses to the microprocessor, whereby when the microprocessor receives each of the electrical impulses of the subject's lung from the means for conducting, the impulse is processed by the signal processor to create a waveform pattern that represents the patient's lung, whereby that pattern is repeatedly compared to patterns stored within the pattern recognition processor. The system further comprises a screen onto which the diagnosis is displayed. The system also comprises a display onto which a computer generated image of the subject's lung is shown identifying any areas of abnormal airway smooth muscle and a display onto which a two dimensional waveform (EPG waveform) representing the electrical impulses of the patient's lung is shown. Signal processing also involves removal of extraneous signal e.g. from the electrocardiogram which will be recorded as routinely performed. Such extraneous noise will be minimized by the use of electrophysiology catheters with small bipoles (thereby limiting the space from which the signal will be recorded): this feature enables the EPG to be physically localized.

In one embodiment, the microprocessor of the system further comprises a processor for generating EPG waveforms. In another embodiment, the pattern recognition processor further comprises a database of patterns representing subjects who have at least some form of abnormal airway contractility and no abnormal airway contractility, such that when a subject's pattern is created by the signal processor, the pattern is compared to other patterns in the database and an assignment of normal or abnormal can then be estimated.

In one embodiment, the abnormal airway electrophysiology and/or abnormal rhythmic contractility signify an asthmatic phenotype and predisposes to asthmatic type airway constriction. In another embodiment, the means for recording airway electrophysiology comprises an electrophysiology catheter that is applied to different parts of the airway under direct visual control via the instrument channel of a flexible bronchoscope-type of device. Signal thus recorded is fed to microchips and microprocessor for signal processing.

In an additional embodiment, the airway electrophysiology signal has characteristic wave morphology, spatial localization and propagation within the airways features that are representative of the subject's airways and may differ between normal subjects, asthmatic subjects and those in an asthma attack. The airway electrophysiology signal is anticipated to arise principally from the airway smooth muscle but may also emanate from the airway epithelium and/or adjacent airway neural structures.

### Advantages of the Invention

In the established paradigm, asthma is primarily a problem of inflammation (and allergy) somewhat analogous to rheumatoid arthritis. However, in the inventor's paradigm, asthma is primarily a problem of aberrant airway pacemaker activity analogous to cardiac dysrhythmias.

In the established paradigm, diagnosis remains clinical with supporting evidence from tests. However, in the inventor's paradigm, definitive diagnosis can be achieved by identifying characteristic airway electrophysiological disturbances.

In the established paradigm, treatment is palliative, i.e. largely still dependent on disease modified by steroids with bronchodilators for acute episodes and waiting for the disease to burn out. However, in the inventor's paradigm, treatment is curative with electrophysiology guided ablation of the aberrant pacemakers or their pathways (like for cardiac arrhythmias).

In the established paradigm, asthma sufferers are resigned to long term care and high financial costs for long term care. However, in the inventor's paradigm, asthma patients are allowed possibility of early cure and costs principally for acute diagnosis and treatment.

### EXAMPLES

### Experimental Methods and Results

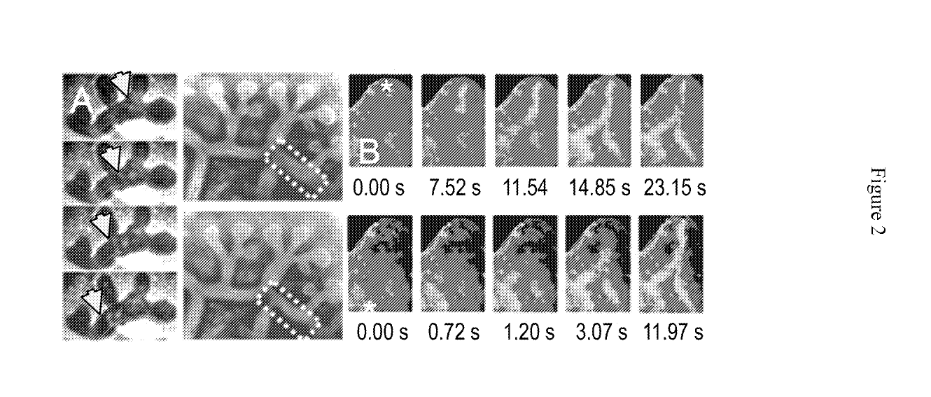

The inventor's airway smooth muscle (ASM) research focused on prenatal ASM and the potential for its contractility to regulate lung growth and discovered phasic contractility (airway peristalsis, (AP)) (FIG. 2) is underpinned by spontaneous propagating calcium waves that require both intracelluar and extracellular calcium and emanate from pacemaker regions in the proximal airway.

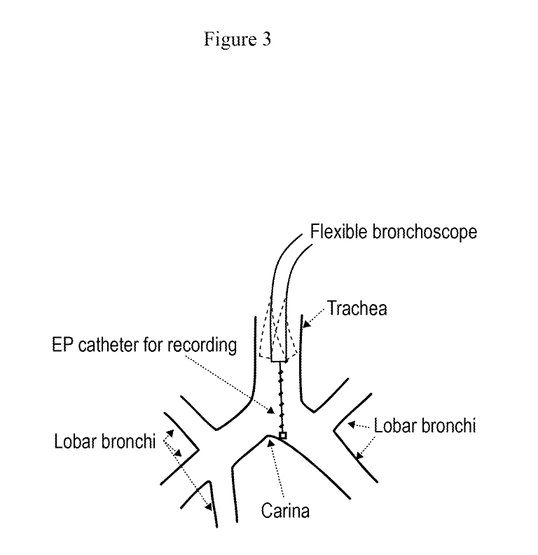

In order to demonstrate airway electrophysiology postnatally in vivo, flexible bronchoscopy on anesthetized pigs was performed and the electrical signals from different parts of the airway were measured by means of a multi-polar cardiac electrophysiology catheter (FIG. 3). The catheter was passed through the instrument channel of the bronchoscope. We advanced the catheter under direct bronchoscopic visualization that allowed the specific catheter electrodes to be applied directly to the airway wall from within the lumen. This method has allowed the Applicant to specify precisely where the recordings were taken from, to confirm good contact with the airway surface and to observe for any respiratory excursions. A portable pacemaker interrogator device (St. Jude, Merlin Interrogator, Model 3650) was used to obtain the recordings and also to pace the airways. Any instrument with similar functionality may be used. The interrogator device was most effective for signal capture.

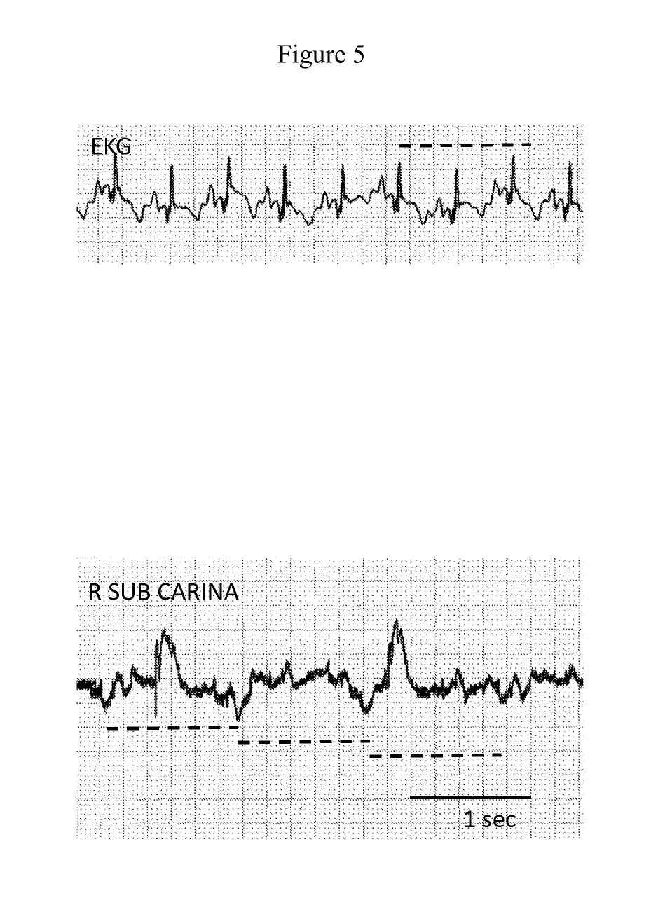

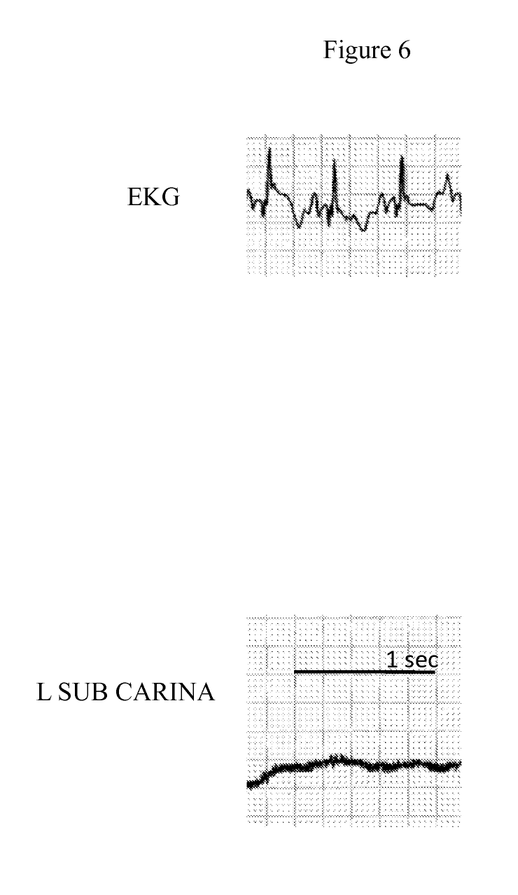

Applicant shows that it is readily possible to obtain clean electrophysiology recordings from the trachea, the main bronchi, the sub-bronchi and the smaller airways. The narrow width of the trace confirms good contact between the electrode and the airway surface. Where the trace width widens, the inventor was able to confirm that the chief cause was the catheter coming away from the surface. This is easily remedied by replacing the catheter tip under direct vision using the bronchoscope. This simple troubleshooting allows good ‘narrow’ traces to be obtained without undue practice. Recordings are performed for between 2 and 15 minutes with longer recordings wherever and whenever there is identification of non-cardiac, non-respiratory rhythms. We were able to confirm that normal breathing rhythms had little or no effect on the trace and supported this by observing that the catheter and airway did not move much relative to one another during the normal breathing cycle. The latter is readily confirmed by briefly halting the respiratory ventilation. We were also able to pick up the electrocardiogram from within the airway. However, as our recordings show our full electropulmonogram comprised separate components too that do not follow the heart rhythm or that of breathing movements (FIG. 5). This provided proof of principle that there is measurable electrical activity in the normal airway in vivo. Furthermore, we found that the signal from different parts of the airway varied in morphology (compare FIGS. 5 and 6). This supports our observation in prenatal airway where we found that most contractile activity emanates from the proximal right lung, designating this a pacemaker zone relative to neighboring regions. Therefore the in vivo electrophysiology (FIG. 5 versus FIG. 6) provides proof of principle that our prenatal finding is relevant to the postnatal scenario, with the proximal right lung being more active than the proximal left lung. This in vivo observation supports the thesis that there are regions within the lung that have greater propensity for pacing.

Therefore, the data presented herein shows for the first time that an electropulmonogram of the lung may be obtained and electrical impulses measured. Similar electropulmonograms from asthmatic subject can be obtained by the same method and the activity compared with normal to distinguish if the region of maximal activity (pacemaker zone) is altered and if the activity itself is changed in character. The latter can be distinguished by analysis of the morphology of the electropulmonogram using the frequency of spontaneous depolarization/hyperpolarisation and the morphology of the waveform (for example, the speed of depolarization, time to half-maximal depolarization/hyperpolarisation, the extent of any plateau phase, the speed of return of polarization to baseline, and/or the speed and size of any pacemaker current; further measures of wave morphology are given in Table 1 from the inventor's publication). The differences in the frequencies of spontaneous depolarization/hyperpolarisation and the various morphological characteristics of the waveform will be apparent to a person of skill in the art (see Table 1).

TABLE 1
TEMPORAL CHARACTERISTICS OF Ca²⁺ OSCILLATIONS
RECORDED AT 37° C. AND 27° C.

| | 37° C. (n = 5) | 27° C. (n = 5) | P Values |
|---|---|---|---|
| Fast phase duration (s) | 0.68 ± 0.05 | 0.92 ± 0.08 | 0.027 |
| Fast phase amplitude (% of | 73.06 ± 1.49 | 75.83 ± 2.33 | NS |
| peak) | | | |
| Time to 50% amplitude (s) | 0.45 ± 0.03 | 0.57 ± 0.06 | NS |
| Slow phase duration (s) | 1.06 ± 0.12 | 1.18 ± 0.17 | NS |
| Plateau duration (s) | 1.94 ± 0.15 | 3.32 ± 0.27 | 0.002 |
| Relaxation half time, T₅₀ | 2.76 ± 0.44 | 4.57 ± 0.33 | 0.01 |
| (s) | | | |
| Width at 50% amplitude (s) | 5.99 ± 0.56 | 9.44 ± 0.54 | 0.002 |

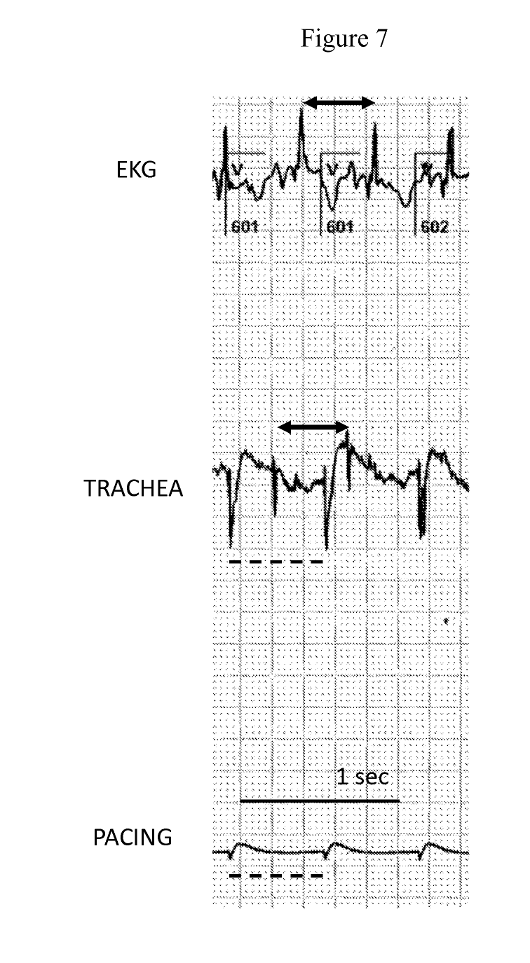

In addition to baseline recordings, we used the multipolar catheter and the pacemaker interrogator to deliver signal to specific parts of the airway and then record the airway signal distal to that. We confirmed that our signal can be transmitted to the airway lower down, in just the manner that we conceive that endogenous pacemakers in the proximal airway signal to distal airways (FIG. 7).

This use of the electrophysiology catheter passed via the flexible bronchoscope to allow recordings at precise locations under direct visualization within the airways is termed ‘spatial mapping’. Spatial mapping permits determination of the optimal sites for placement of the implanted electrode tips for temporal mapping. The latter is where electrodes are secured thoracoscopically on the external surface of the larger airways which when connected to an implanted pacemaker with recording modes allows long term study during daily living (temporal mapping).

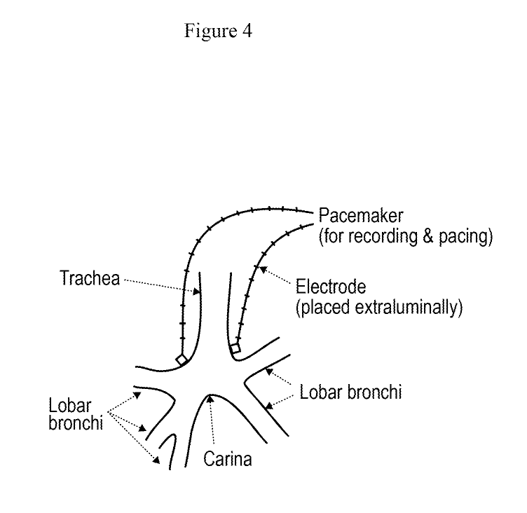

Temporal mapping (FIG. 4 and below) facilitates capture of longer-term periodicities. Anaesthetised pigs undergo surgery to implant subcutaneously a standard remotely programmable commercially available pacemaker device that permits both EP recording and pacing. This is the standard method in use for subcutaneous implantation of cardiac pacemakers in humans. However, in this case, rather than the electrodes being routed to the heart via the vasculature, the electrode catheter tips are tunneled via the extrapleural space using standard thoracoscopic visualization and instrumentation to place them on the external surface of the proximal airways. These relevant anatomical positions are readily visible on thoracoscopy in exactly the same way that surgeons access the main airways to perform thoracoscopic lung or lobe resections. The animals are then recovered and continuous EP recordings obtained via the sensing mode of the implanted device.

The following technical variations can be pursued: percutaneous peritracheal electrode placement under image guidance; transesophageal or even transcutaneous measurement. Although technically demanding, transcutaneous recording is performed for the stomach (electrogastrography) and has even been used to measure individual fetal electrocardiograms during twin and triplet pregnancies using only surface electrodes attached to the maternal abdominal skin.

EP-guided radiofrequency ablation to prevent the asthma-style small airway constriction will use two approaches based on techniques for electrophysiological ablations in atrial dysrhythmias: the first is direct ablation of any aberrant pacemaker focus in proximal airway; the second involves creating a barrier to prevent signal conduction between proximal and distal airways. With both methods, the proximal airway EP activity is disconnected from the small airways that constrict during typical asthma attacks. This approach avoids the indiscriminate, time-consuming and costly need to apply bronchial thermoplasty to each and every accessible airway.

Use of temporal mapping allows fluctuations in airway EP before and after asthma attacks to be monitored in order to identify individual triggers that can be used to alert the sufferer (c.f. cardiac pacemakers that trigger automatically in the event of impending or actual dysrhythmia). Rendering ASM ‘safe’ and refractory is achieved by, for example, chronic mild depolarization via implanted EP catheter electrode.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method for diagnosing asthma in a subject in need thereof comprising:
(i) obtaining an electropulmonogram illustrating electrical impulses from one or more lungs of the subject by:
(a) inserting a flexible bronchoscope in the one or more lungs of the subject;
(b) inserting an electrophysiology catheter through the bronchoscope; and
(c) measuring the electrical impulses from the one or more lungs using the electrophysiology catheter; and
(ii) analyzing the frequency of spontaneous depolarization and/or hyperpolarization and/or morphology of the waveform,
wherein differences in the frequency of spontaneous depolarization and/or hyperpolarization and/or morphology of the waveform between the subject and a control subject is indicative of asthma in the subject, so as to diagnose asthma in the subject.

2. A method for diagnosis of abnormal rhythmic peristalsis in a subject in need thereof comprising:
(i) obtaining an electropulmonogram illustrating electrical impulses from one or more lungs of the subject comprising:
(a) inserting a flexible bronchoscope in the one or more lungs of the subject;
(b) inserting an electrophysiology catheter through the bronchoscope; and
(c) measuring the electrical impulses from the one or more lungs using the electrophysiology catheter; and
(ii) analyzing the frequency of spontaneous depolarization and/or hyperpolarization and/or morphology of the waveform,
wherein differences in the frequency of spontaneous depolarization and/or hyperpolarization and/or morphology of the waveform between the subject and a control subject is indicative of abnormal rhythmic peristalsis in the subject, so as to abnormal rhythmic peristalsis in the subject.

3. A method for detecting abnormal pacemakers in the lung in a subject in need thereof comprising:
(i) obtaining an electropulmonogram illustrating electrical impulses from one or more lungs of the subject comprising:
(a) inserting a flexible bronchoscope in the one or more lungs of the subject;
(b) inserting an electrophysiology catheter through the bronchoscope; and
(c) measuring the electrical impulses from the one or more lungs using the electrophysiology catheter; and
(ii) analyzing the frequency of spontaneous depolarization and/or hyperpolarization and/or morphology of the waveform,
wherein differences in the frequency of spontaneous depolarization and/or hyperpolarization and/or morphology of the waveform between the subject and a control subject is indicative of abnormal pacemakers in the lung in the subject.

4. A method for detecting asthma in a subject in need thereof by the methods of claim 2, wherein presence of abnormal rhythmic peristalsis or abnormal pacemakers in the subject is indicative of asthma in the subject.

5. A method for treating asthma in a subject in need thereof comprising:
(i) detecting abnormal pacemakers comprising obtaining an electropulmonogram illustrating electrical impulses from one or more lungs of the subject comprising:
(a) inserting a flexible bronchoscope in the one or more lungs of the subject;
(b) inserting an electrophysiology catheter through the bronchoscope; and
(c) measuring the electrical impulses from the one or more lungs using the electrophysiology catheter;
(ii) analyzing the frequency of spontaneous depolarization and/or hyperpolarization and/or morphology of the waveform, wherein differences in the frequency of spontaneous depolarization and/or hyperpolarization and/or morphology of the waveform between the subject and a control subject is indicative of abnormal pacemakers in the lung in the subject; and
(iii) inserting a thermal ablation catheter through the flexible bronchoscope to target the abnormal pacemaker; and
(iv) using thermal ablation to reduce the activity of the abnormal pacemakers or destroy abnormal pacemakers,
so as to treat asthma in the subject.

6. A method for treating asthma in a subject in need thereof comprising:
(i) detecting abnormal rhythmic peristalsis in the subject comprising obtaining an electropulmonogram illustrating electrical impulses from one or more lungs of the subject comprising:
(a) inserting a flexible bronchoscope in the one or more lungs of the subject;
(b) inserting an electrophysiology catheter through the bronchoscope; and
(c) measuring the electrical impulses from the one or more lungs using the electrophysiology catheter;
(ii) analyzing the frequency of spontaneous depolarization and/or hyperpolarization and/or morphology of the waveform, wherein differences in the frequency of spontaneous depolarization and/or hyperpolarization and/or morphology of the waveform between the subject and a control subject is indicative of abnormal rhythmic peristalsis in the subject;
(iii) inserting a radiofrequency catheter through the flexible bronchoscope to target abnormal airway smooth muscle; and
(iv) destroying the abnormal smooth muscle focally using radiofrequency ablation to render airway smooth muscle quiescent, so as to treat the asthma in the subject.

7. A method for diagnosing asthma in a subject in need thereof comprising:
(i) obtaining electrical impulses produced by one or more lungs of the subject using a means for sensing the electrical impulses;
(ii) processing and multiplexing the electrical impulses received from the means for sensing to create a two dimensional waveform; and
(ii) comparing the two dimensional waveform to other waveforms contained within a database in a microprocessor, wherein the database comprises waveforms of patients that have asthma and waveforms of patients that do not have asthma,
wherein the identity between the subject's waveform and the asthmatic waveform in the database or the difference between the subject's waveform and that non-asthmatic waveform in the database is indicative of asthma in the subject, so as to diagnose asthma in the subject.

8. The method of claim 7, wherein the two dimensional waveform is an electropulmonogram.

9. The method of claim 7, further comprising displaying on a screen a computer generated image of the one or more lungs of the patient in which areas of abnormal airway electrophysiology are identifiable.

10. The method of claim 7, further comprising displaying on a screen the two dimensional waveform.

11. A system for testing a subject having one or more lungs for abnormal airway pacemakers and/or abnormal rhythmic airway contractility comprising:
(i) a microprocessor comprising a signal processor and a pattern recognition processor; and
(ii) a means for conducting electrical impulses of the one or more lungs and communicating the electrical impulses to the microprocessor, wherein when the microprocessor receives each of the electrical impulses, the electrical impulses are processed by the signal processor to create a waveform pattern that represents an airway electrophysiology of the subject, whereby the waveform pattern is repeatedly compared to patterns stored within a database in the pattern recognition processor.

12. The system of claim 11, wherein the subject has asthma.

13. The system of claim 11, wherein the means for conducting the electrical impulses comprises an electrophysiology catheter in communication with the one or more lungs via an instrument channel of a bronchoscope-type device, to obtain electrophysiology recordings from the one or more lungs.

14. The system of claim 11, further comprising a screen onto which a diagnosis of abnormal airway pacemakers and/or abnormal rhythmic airway contractility is displayed.

15. The system of claim 11, further comprising a display onto which a computer generated image of the one or more lungs may be shown and in which areas of abnormal airway smooth muscle are identifiable.

16. The system of claim 11, further comprising a display onto which a two dimensional waveform representing the electrical impulses may be shown.

17. The system of claim 11, wherein the microprocessor further comprises a processor for generating EPG waveforms.

18. The system of claim 11, wherein the database comprises patterns representing subjects who have at least some form of abnormal rhythmic peristalsis and no abnormal rhythmic peristalsis.

19. A method for diagnosing vesicoureteral reflux in a subject in need thereof comprising:
(i) obtaining an electroureterogram illustrating electrical impulses from the ureter comprising
(a) inserting a ureteroscope in the ureter of the subject;
(b) inserting an electrophysiology catheter through the ureteroscope; and
(c) measuring the electrical impulses from the ureter using the electrophysiology catheter; and
(ii) analyzing the frequency of spontaneous depolarization and/or hyperpolarization and/or morphology of the waveform,
wherein differences in the frequency of spontaneous depolarization and/or hyperpolarization and/or morphology of the waveform between the subject and a control subject is indicative of vesicoureteral reflux in the subject.
